(19) United States
(12) Patent Application Publication
(10) Pub. No.: US 2006/0094962 A1
(43) Pub. Date: May 4, 2006

(54) Aperture shading estimation techniques for reducing ultrasound multi-line image distortion

(76) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(21) Appl. No.: 11/229,158

(22) Filed: Sep. 16, 2005

Related U.S. Application Data
(60) Provisional application No. 60/623,746, filed on Oct. 29, 2004.

(51) Int. Cl. A61B 8/06

(52) U.S. Cl. 600/447

(57) ABSTRACT

In an ultrasound imaging system, line-to-line image distortion is reduced by estimating aperture shading caused by the presence of an occlusion in a test subject. A plurality of transmit beams are transmitted towards a subject using an ultrasound transmitter having an aperture (201). Each transmit beam is associated with a plurality of receive beams that are reflected by the subject. Receive beams are collected using a plurality of receive channels (203). For each receive channel, receive data is derived from one or more collected receive beams (204), and a sum of the absolute values of the receive data is generated (205). The sums of the absolute values are smoothed and normalized for the set of receive channels (207), so as to generate an estimate for a shaded aperture function that characterizes aperture shading caused by the occlusion. The shaded aperture function is utilized as a receive apodization function to improve the signal-to-noise ratio of the receive data (209). The centroid of the shaded aperture function is also utilized to align the receive steering and focusing to the transmit beam steering effects of the occlusion.

The present invention relates generally to ultrasound imaging and, more particularly, to methods for reducing multi-line image distortion in medical diagnostic ultrasound imaging.

Ultrasound, also referred to as diagnostic medical sonography or echocardiography, is an imaging method that utilizes high-frequency acoustical waves to produce images of structures within the human body. These images provide information that is useful in diagnosing and guiding the treatment of disease. For example, ultrasound is frequently employed during pregnancy to determine the health and development of a fetus. Ultrasound is also used as a diagnostic aid for recognizing subtle differences between healthy and unhealthy tissues in organs of the neck, abdomen and pelvis. It is also very useful in locating and determining the extent of disease in blood vessels. Echocardiography—ultrasound imaging of the heart—is used to diagnose many heart conditions. Accurate biopsy and treatment of tumors is facilitated through the use of ultrasound guidance procedures which provide images of healthy tissues in proximity to the tumor.

Conventional medical sonography is conducted with the use of diagnostic ultrasound equipment that transmits acoustical energy into the human body and receives signals that are reflected by bodily tissues and organs such as the heart, liver, and kidneys. The motion of blood cells causes Doppler frequency shifts in the reflected signals. In the time domain, these frequency shifts are observed as shifts in cross-correlation functions of the reflected signals. The reflected signals are typically displayed in a two-dimensional format known as color flow imaging or color velocity imaging. Such displays are commonly utilized to examine blood flow patterns. A typical ultrasound system emits pulses over a plurality of paths and converts echoes received from objects on the plurality of paths into electrical signals used to generate ultrasound data from which an ultrasound image can be displayed. The process of obtaining raw ultrasound data from which image data is produced is typically termed “scanning,” “sweeping,” or “steering a beam”.

Sonography may be performed in real time, which refers to a rapid, sequential presentation of ultrasound images as scanning is performed. Scanning is usually performed electronically, utilizing a group of transducer elements (called an “array”) arranged in a line and excited by a set of electrical pulses, one pulse per element for each of a plurality of cyclic sequences. Pulses are typically timed to construct a sweeping action throughout a diagnostic region to be imaged.

Signal processing in an ultrasound scanner commences with the shaping and delaying of the excitation pulses applied to each element of the array so as to generate a focused, steered and apodized pulsed beam that at least partially propagates into human tissue. Processing is typically performed at the individual element level, or at the channel level, wherein a channel includes one or more elements. Apodization refers to a process of tapering channel amplitudes using a weighting function. The characteristics of the transmitted acoustic pulse may be adjusted or “shaped” to correspond to the setting of a particular imaging mode. For example, pulse shaping may include adjusting the length of the pulse depending on whether the returned echoes are to be used in B-scan, pulsed Doppler or color Doppler imaging modes. Pulse shaping may also include adjustments to the pulse frequency which, in modern broadband transducers, can be set over a wide range and may be selected according to the part of the body that is being scanned. A number of scanners also shape the envelope of the pulse (i.e., a Gaussian envelope) to improve the propagation characteristics of the resulting acoustical wave.

Echo signals resulting from scattering of the acoustical wave by tissue structures are received by all of the elements within the transducer array and are subsequently processed. The processing of these echo signals is typically performed at the individual element level, or at the channel level, wherein a channel includes one or more elements. Signal processing commences with the application of apodization functions, dynamic focusing, and steering delays. One of the most important elements in signal processing is beam formation. In a transducer array, the beam is focused and steered by exciting each of the elements at different times such that the acoustical wave transmitted by each element will arrive at an intended focal point simultaneously with the arrival of acoustical waves from all of the other elements in the array.

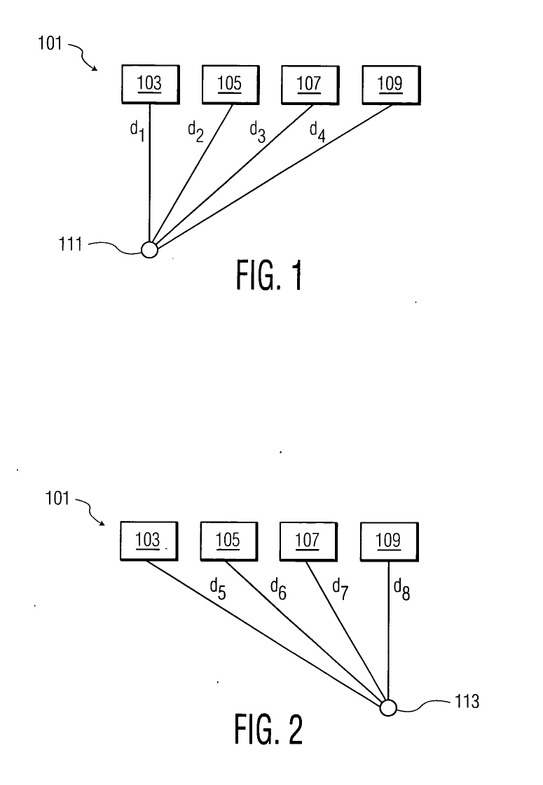

Beam focusing and steering is better understood with reference to FIG. 1, depicting a transducer array 101 having transducers 103, 105, 107 and 109 at distances d₁, d₂, d₃ and d₄, respectively, from a focal point 111. In the present example, the beam is to be focused and steered to the left. The distance d₁ from focal point 111 to transducer element 103 of transducer array 101 is shorter than the distance d₄ from focal point 111 to transducer element 109. Accordingly, during transmission, element 109 must be excited before elements 103, 105, and 107 in order for the waves generated by each element to arrive at the focal point simultaneously. FIG. 2 shows the manner in which transducer array 101 may be used to establish a focal point 113 to the right. Here, the elements of transducer array 101 must be excited in the reverse order during transmission with reference to the example of FIG. 1. In other words, element 103 must be excited before elements 105, 107, and 109 in order for the waves generated by each element to arrive at focal point 113 simultaneously. This process of coordinating the firing of transducer elements is referred to as “beam formation”, and the device which implements this process is called a “beamformer”.

Beam forming is typically implemented during both transmission (described above) and reception. Beam forming on reception is conceptually similar to beam forming on transmission. On reception, an echo returning from a given point, such as focal point 111 (FIG. 1), encounters each of the elements 103, 105, 107 and 109 in the transducer array 101 at a different time due to the varying distances d₁, d₂, d₃ and d₄, respectively, of these elements from focal point 111. Consequently, the signals coming into the ultrasound scanner from the various elements 103, 105, 107, 109 must be delayed so that they all “arrive” at the ultrasound scanner at the same moment. The signals from each element 103, 105, 107, 109 are then summed together to form the ultrasound signal that is subsequently processed by the rest of the ultrasound scanner. Typically, transducer array 101 is implemented using a one-dimensional array having 32 to 192 transducer elements for beam formation. The signal from each individual element is delayed in order to steer the beam in a desired direction.

In addition to combining the received signals into an output signal, the beamformer also focuses the receive beam. In situations where dynamic focusing is employed, for each pulse which is transmitted from the array, the beamformer tracks the depth and focuses the receive beam as the depth increases. The receive aperture will usually be allowed to increase with depth, since this achieves a lateral resolution which is constant with depth, and advantageously decreases sensitivity to aberrations in the imaged medium. In order for the receive aperture to increase with depth, it is necessary to dynamically control the number of elements in the array used to receive echoes in the form of receive beams. An apodization process applies weighting functions to taper the amplitudes of a plurality of channels. The element weights may be dynamically updated with depth.

Many ultrasound scanners are able to perform parallel beam forming. Parallel beam forming refers to the acquisition of multiple, round-trip receive beams from a single transmit beam by focusing a plurality of receive beams derived from the single transmit beam at each of a plurality of scan lines. Multiple beamformers, working in parallel, provide simultaneous reception of several scan lines after one transmission pulse. The transmit scan line is wide enough to accommodate a group of, for example, four receive scan lines. The receive scan lines are created by electronic focusing, whereby the multiple beamformers each implement slightly different delays of the receive beams as appropriate for each receive scan line. The time savings achieved by generating several receive scan lines simultaneously can be used to increase the image frame rate, to increase lateral resolution by allowing more scan lines at a given frame rate, and to increase flow velocity sensitivity and resolution in color Doppler sonography by allowing more time at each Doppler scan line.

The transmit beam, due to its single focus, is typically apodized to improve depth of field and is therefore inherently wider than each of a plurality of dynamically focused receive beams. The receive beams have local acoustical maxima which are off-axis relative to the transmit beam. Parallel beam forming allows an imaged field to be scanned faster, thereby allowing faster updating of image frames. Parallel beam forming is especially advantageous in 3-D imaging, due to the large number of frames that must be gathered.

While parallel beam forming has many notable advantages, its application is significantly complicated by anatomical features. For example, during the imaging of myocardial tissues, the aperture of the phased array transducer is often partially blocked by a rib, thus causing a portion of the aperture to be shaded. Consequently, the resulting transmit beam shifts in location, pivoting around the transmit focal point, while the receive beam continues to track its original beam location because it is continually focused at all depths. This effect causes the roundtrip beam pattern to lose amplitude at all depths other than the transmit focal depth. Moreover, during parallel beam forming, aperture shading affects each of the parallel roundtrip beams (each parallel roundtrip beam representing a given line) differently, thereby creating “line-to-line” amplitude modulation distortions in the image. These distortions, also termed “artifacts”, cause variations in image brightness which appear as annoying striations running across the image when those beams are placed side by side. In conventional imaging schemes, this problem is partially ameliorated through the use of lateral blending filters that reduce the amplitude differences between lines to produce more uniform brightness. However, this approach significantly degrades image resolution.

It is possible to estimate the centroid of a shaded aperture by measuring the relative average amplitudes of a plurality of receive beams, and controlling the beamformer to shift the path of the receive beams in accordance with the shaded portion of the aperture. One or more receive channels that are deemed to be shaded are disabled to improve the signal-to-noise ratio of the non-disabled receive channels. Aperture shading compensation is applied at the beamformer, and the centroid of the shaded aperture is estimated from feedback in the form of receive data. As will be described in greater detail hereinafter, what is estimated is not the centroid of the shaded aperture, but rather the current error in the immediately preceding estimate. Since feedback is acquired after the paths of the receive beams have been shifted by the beamformer, precautions must be adopted to ensure that such feedback does not cause the beamformer to become unstable.

Estimating aperture shading by measuring the relative average amplitudes of the receive beams is a complicated procedure. At each of a plurality of transmit focal depths, for each of a plurality of transmit beams, the centroid of the data from the multiple receive beams is calculated relative to the center focus of the plurality of transmit beams. This calculation is averaged over all transmit beams used to produce an image, and converted into an angular error for that transmit focal depth, relative to the center focus. The angular errors are weight-averaged over a depth range to obtain an overall angular error, employing a weighting equal to the distance from the center focus. This effectively fits a straight line pivoted on the transmit focus to the average receive centroid errors as a function of receive focal depth.

An error centroid of the shaded aperture is calculated from the overall angular error and the transmit focal depth. Unfortunately, this approach provides many sources of error. The detailed shape of the aperture shading function is not estimated, only the current error in its centroid relative to the assumed position from the immediately preceding estimate. Consequently, an aperture shading function must be assumed or guessed in order to be applied as a receive apodization for improving the signal to noise ratio of the non-disabled receive channels. Moreover, there is inevitable bias and variance in the receive data centroids due to non-uniformity of the structures being imaged, and also due to speckle and noise. These errors are generally magnified when projected back to the aperture through the transmit focus.

There is thus a need in the art for a method for effectively reducing or eliminating “line-to-line” amplitude modulation image distortion and other image distortion without sacrificing image resolution. There is also a need in the art for such methods that can be employed statically or dynamically. These and other needs are met by the methodologies and devices disclosed herein.

In an ultrasound imaging system, line-to-line image distortion is reduced by estimating aperture shading caused by the presence of an occlusion in a test subject. A compensation technique then compensates for the presence of the occlusion.

Pursuant to one embodiment of the invention, a plurality of transmit beams are transmitted toward a subject using an ultrasound transmitter having an aperture. Each transmit beam is associated with a plurality of receive beams that are reflected by the subject. Receive beams are collected using a plurality of receive channels. For each receive channel, receive data is derived from one or more collected receive beams, and a sum of the absolute values of the receive data is generated. The sums of the absolute values are smoothed and normalized for each receive channel, so as to generate an estimate for a shaded aperture function that characterizes aperture shading caused by the occlusion. The shaded aperture function is utilized as a receive apodization function to improve the signal-to-noise ratio of the receive data. Apodization refers to a process of tapering the receive channel amplitudes using a weighting function.

A further embodiment of the invention compensates for the occlusion by using the shaded aperture function to determine a first centroid for the aperture. Based upon the shaded aperture function, one or more receive channels and one or more transmit channels associated with an occluded portion of the aperture are deactivated. Receive channels are deactivated for beamforming and imaging, but continue to be used for aperture shading estimation. A second centroid for the aperture is then determined. Using the second centroid, the paths of any receive channels that have not been deactivated are adjusted by determining each of a plurality of respective magnitude scaling factors and delays to be applied to each of a plurality of corresponding receive channels. The respective magnitude scaling factors and delays are determined such that a center channel focusing coefficient specifying the center focus of the receive channels that have not been deactivated is aligned with the second centroid. The foregoing procedure is performed either statically or dynamically. Optionally, the one or more channels to be deactivated are determined by measuring the magnitudes and locations of amplitude modulation components on each of the receive channels as a function of placement of the receive beams, by monitoring each receive beam, and/or by transmitting one or more calibration beams.

Pursuant to another further embodiment of the invention, the step of compensating for the occlusion is performed by moving the centroid of the aperture from a first location to a second location. Each of the receive channels is monitored, preferably in real time, to identify any aperture elements that are shaded by an occlusion. The receive channels associated with any aperture elements shaded by an occlusion are deactivated for beamforming and imaging (although still used for continued aperture shading estimation), and a focal point of the receive beams is re-aligned such that at least one receive channel is aligned with the first location. Optionally, the amount and location of amplitude modulation components relative to placement of the receive beams is employed as an indicator of the amount and location of aperture shading.

Pursuant to another embodiment of the invention, an ultrasound imaging device is provided which estimates the extent of any occlusion of the aperture, and then compensates for the estimated occlusion. The imaging device comprises a transducer array, an analog to digital converter, a bandpass or highpass filter, an absolute value extractor, a smoothing mechanism, a system control mechanism, a channel normalization mechanism, and a receive beamformer. The transducer array, having an aperture associated therewith, emits acoustic pulses over a plurality of transmit channels, and receives analog echoes of these pulses over a plurality of receive channels. The transducer array is coupled to the analog to digital converter which converts the analog echoes into digital receive data. The digital receive data which are used for aperture shading estimation are filtered by the bandpass or highpass filter to deemphasize lower frequencies, after which the absolute value extractor extracts absolute values for the filtered digital receive data. The extracted absolute values are smoothed by the smoothing mechanism. The normalization mechanism normalizes the smoothed extracted absolute values from a plurality of receive channels to generate an estimate of the extent of any occlusion of the aperture in the form of an apodization function. The apodization function improves the signal-to-noise ratio of the receive data. The normalization mechanism then determines the centroid of the apodization function. The centroid is utilized in conjunction with a beamformer to adjust the paths of one or more of the receive beams, so as to compensate for the occlusion.

The beamformer may be adapted to (a) monitor each of the receive channels so as to determine the extent of any occlusion that moves the original center of the aperture to a new center, (b) deactivate the receive channels associated with any aperture elements blocked by an occlusion, and (c) re-align the receive focusing such that the center receive channels are aligned with the new center of the aperture.

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter in which there are illustrated and described preferred embodiments of the invention.

In the drawings:

For a more complete understanding of the present invention and advantages thereof, reference is now made to the following description which is to be taken in conjunction with the accompanying drawings in which like reference numbers indicate like features and wherein:

FIG. 1 is a diagram illustrating the need for time delay to account for differences in the distances between the elements of a transducer array and a focal point in an ultrasound diagnostic system;

FIG. 2 is a diagram illustrating the need for time delay to account for differences in the distances between the elements of a transducer array and a focal point in an ultrasound diagnostic system;

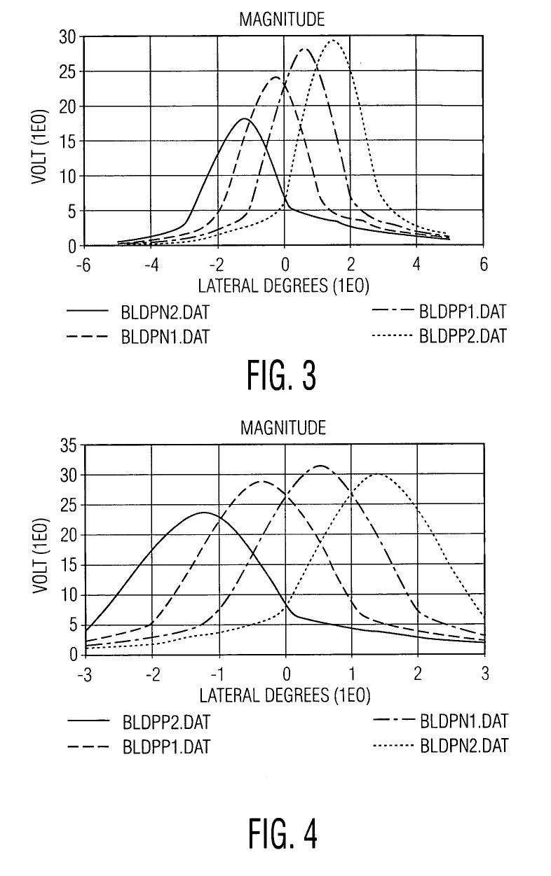

FIG. 3 is a roundtrip beam profile for a 4-way parallel situation and in which a rib blocks the right ⅓ of the total physical aperture;

FIG. 4 is a roundtrip beam profile for a 4-way parallel situation and in which a rib blocks the right ¼ of the total physical aperture;

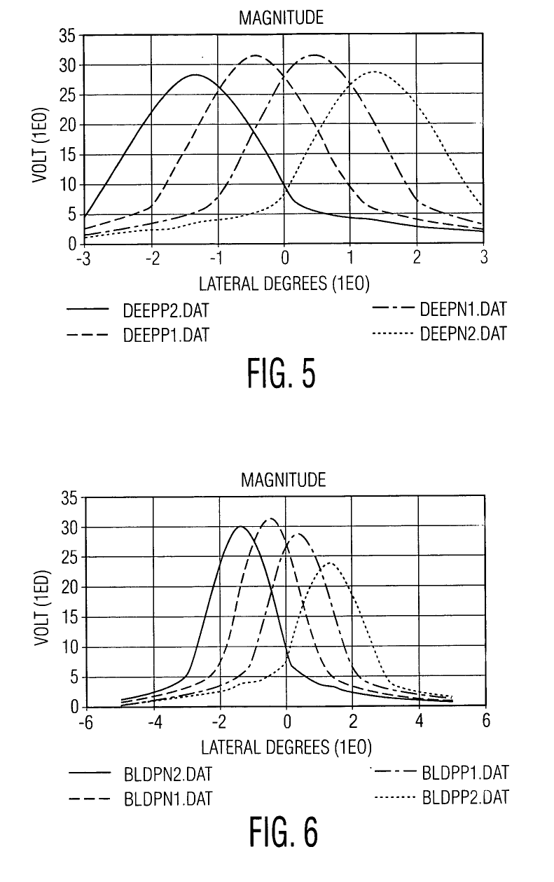

FIG. 5 is a roundtrip beam profile for a 4-way parallel situation with no rib blockage;

FIG. 6 is a roundtrip beam profile for a 4-way parallel situation and in which a rib blocks the left ¼ of the total physical aperture;

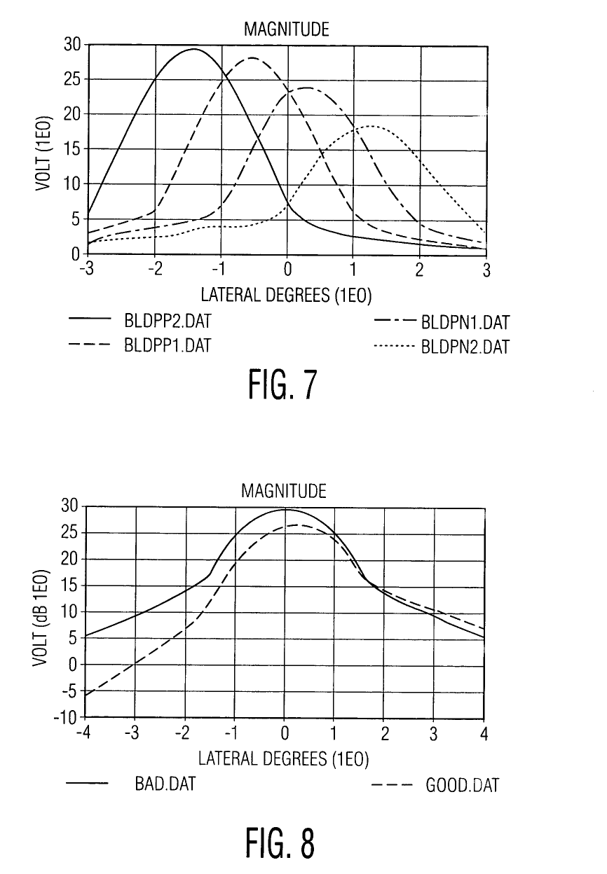

FIG. 7 is a roundtrip beam profile for a 4-way parallel situation and in which a rib blocks the left ⅓ of the total physical aperture;

FIG. 8 is a roundtrip beam profile for a non-parallel case illustrating the drop in amplitude of the roundtrip beam away from the transmit focus and the creation of an asymmetric side lobe pattern;

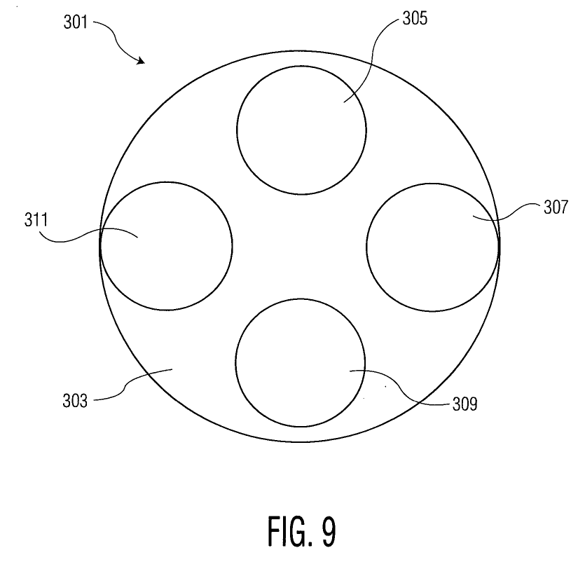

FIG. 9 is an illustration of a 4-way parallel beam pattern on the receive side;

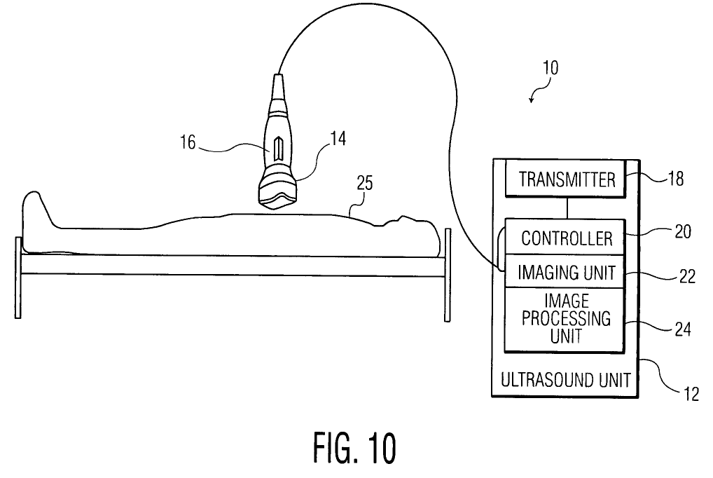

FIG. 10 is an illustration of an ultrasound device which may be used to implement the methodologies disclosed herein;

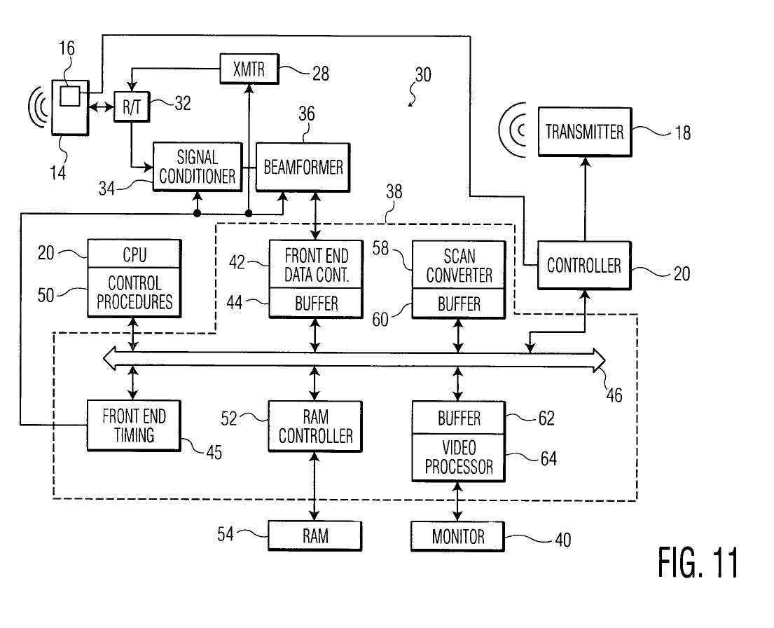

FIG. 11 is a schematic diagram illustrating the functional elements of a device of the type depicted in FIG. 10;

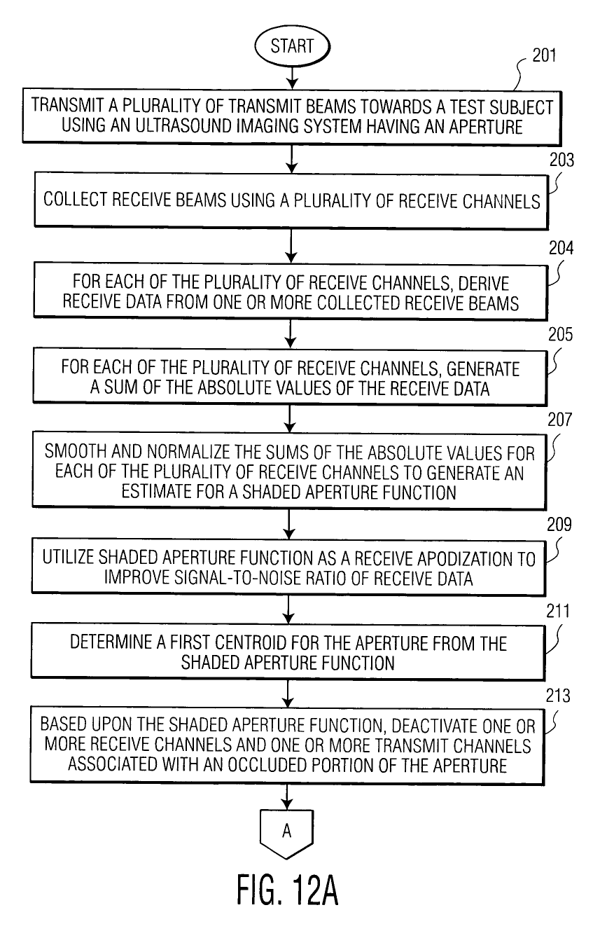

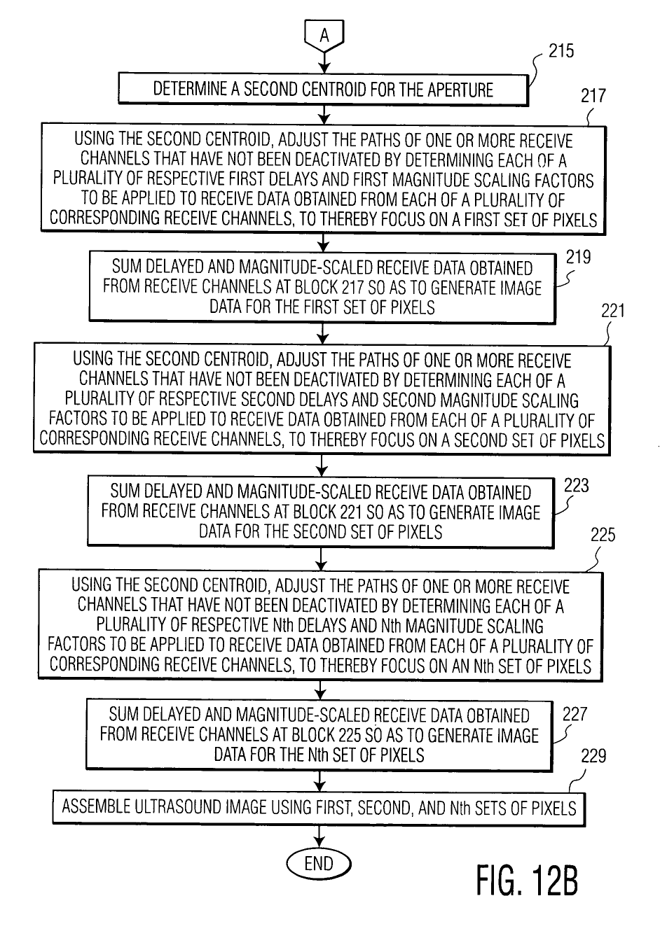

FIG. 12 sets forth an operational sequence for estimating and providing compensation for an aperture occlusion according to a preferred embodiment of the invention; and

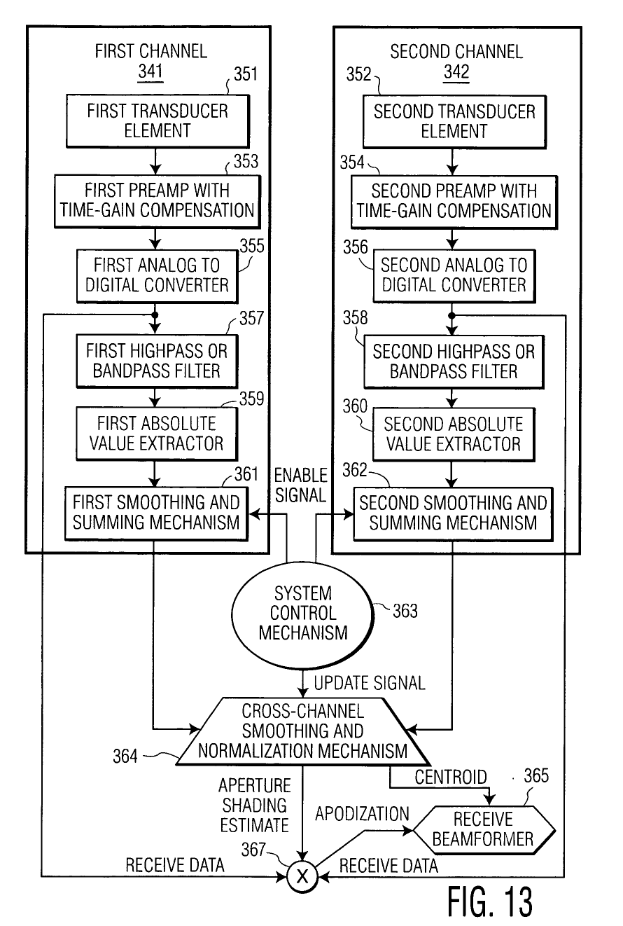

FIG. 13 is a hardware block diagram of an exemplary apparatus for performing the operational sequence of FIG. 12.

Various preferred embodiments of the present invention and its advantages are best understood by referring to FIGS. 1 through 13, like numerals being used for like and corresponding parts throughout the various drawings.

In an ultrasound imaging system, line-to-line image distortions are reduced by estimating aperture shading caused by the presence of an occlusion in a test subject. A compensation scheme then compensates for the presence of the occlusion.

FIG. 12 sets forth an operational sequence for estimating and providing compensation for an aperture occlusion according to a preferred embodiment of the invention. The sequence commences at block 201 where a plurality of transmit beams are transmitted toward a test subject using an ultrasound imaging system having an aperture. Each transmit beam is associated with a plurality of receive beams that are reflected by the subject. At block 203, these receive beams are collected using a plurality of receive channels. For each of the plurality of receive channels, receive data are derived from one or more collected receive beams (block 204), and a sum of the absolute values of the receive data is generated (block 205).

The sums of the absolute values are smoothed and normalized for each receive channel (block 207), so as to generate an estimate for a shaded aperture function that characterizes the extent to which the occlusion shades or blocks the aperture. The shaded aperture function is utilized as a receive apodization function to improve the signal-to-noise ratio of the receive data (block 209). Apodization refers to a process of tapering channel amplitudes using a weighting function.

Compensation for the occlusion is provided by using the shaded aperture function to determine a first centroid for the aperture (block 211). Based upon the shaded aperture function, one or more receive channels and one or more transmit channels associated with an occluded portion of the aperture are deactivated (block 213). Receive channels that are deactivated are not used for beamforming and image formation, but continue to be used for aperture shading estimation. A second centroid for the aperture is then determined (block 215). Using the second centroid, the paths of one or more receive channels that have not been deactivated are adjusted by determining each of a plurality of respective first delays and first magnitude scaling factors to be applied to receive data obtained from each of a plurality of corresponding receive channels (block 217), to thereby focus on a first set of pixels. The respective delays and magnitude scaling factors are determined such that a center channel focusing coefficient specifying the center focus of the receive channels that have not been deactivated is aligned with the second centroid of the aperture. After receive data obtained from each of a plurality of receive channels is time shifted and scaled (block 217), the data are summed so as to generate image data for the first set of pixels (block 219).

Using the second centroid, the paths of one or more receive channels that have not been deactivated are adjusted by determining each of a plurality of respective second delays and second magnitude scaling factors to be applied to receive data obtained from each of a plurality of corresponding receive channels (block 221), to thereby focus on a second set of pixels. The respective delays and magnitude scaling factors are determined such that a center channel focusing coefficient specifying the center focus of the receive channels that have not been deactivated is aligned with the second centroid of the aperture. After receive data obtained from each of a plurality of receive channels is time shifted and scaled (block 221), the data are summed so as to generate image data for the second set of pixels (block 223).

Using the second centroid, the paths of one or more receive channels that have not been deactivated are adjusted by determining each of a plurality of respective Nth delays and Nth magnitude scaling factors to be applied to receive data obtained from each of a plurality of corresponding receive channels (block 225), to thereby focus on an Nth set of pixels. The respective delays and magnitude scaling factors are determined such that a center channel focusing coefficient specifying the center focus of the receive channels that have not been deactivated is aligned with the second centroid of the aperture. After receive data obtained from each of a plurality of receive channels is time shifted and scaled (block 225), the data are summed so as to generate image data for the Nth set of pixels (block 227).

An ultrasound image is assembled using image data for the first, second, and Nth sets of pixels determined, respectively, at blocks 219, 223, and 227. Essentially, the method of FIGS. 12 and 12 assembles an ultrasound image by sequentially focuses on different sets of pixels. The concept of time shifting and scaling receive data acquired from the transducer array on a receive channel provides the ability to “focus” on a set of pixels in a scan region encompassing a test subject. Pursuant to parallel scanning procedures, the first set of pixels may encompass a plurality of lines in the image.

FIG. 13 is a hardware block diagram of an exemplary apparatus for performing the operational sequence of FIG. 12. A first transducer element 351 and a second transducer element 352 are shown for illustrative purposes, it being clearly understood that the configuration of FIG. 13 may be adapted to a two-dimensional array of transducer elements for use in any three-dimensional imaging process. The configuration of FIG. 13 may also be adapted to a one-dimensional array of transducer elements for use in any two-dimensional imaging process. Moreover, the configuration of FIG. 13 may be employed to implement beamforming on any portion of an array of transducer elements, in conjunction with individual elements or groups of elements. Ultrasound acoustical energy received by first transducer element 351 are processed using a first channel 341, and ultrasound acoustical energy received by second transducer element 352 is processed using a second channel 342.

The output of first transducer element 351 is received by a first preamp with time-gain compensation 353, and the output of second transducer element 352 is received by a second preamp with time-gain compensation 354. Time-gain compensation is employed to correct for the decreasing amplitudes of echoes from progressively deeper depths in a test subject. A first analog to digital converter 355 converts analog signals received from first preamp with time-gain compensation 353 into digitized signals. A second analog to digital converter 356 converts analog signals received from second preamp with time-gain compensation 354 into digitized signals. Digitized signals from first analog to digital converter 355 are filtered by a first highpass or bandpass filter 357, and digitized signals from second analog to digital converter 356 are filtered by a second highpass or bandpass filter 358.

Virtually all ultrasound imaging systems include some type of filter which shapes the frequency response of the receive path, generally by attenuating lower frequency echoes and/or by enhancing higher frequency echoes. Pursuant to prior art techniques, such a filter is applied after beamforming. A preferred embodiment of the invention disclosed herein employs another, typically much simpler, filter prior to beamforming for the data that is used to estimate the aperture shading, which advantageously avoids biasing an aperture shading estimate where the biasing is caused by responding too much to lower-frequency echoes that do not affect the image. First and second highpass or bandpass filters 357, 358 are implemented using a first-order successive difference algorithm to be applied to the digitized signals received from, respectively, first and second analog to digital converters 355, 356, respectively. The first-order successive difference algorithm has the effect of greatly attenuating lower frequency components from the digitized signals. Alternatively, an algorithm which utilizes a periodic pattern of adding and subtracting the digitized signals for short periods of time may be employed to implement first and second highpass or bandpass filters 357, 358, thereby providing a filter that passes a band of frequencies. First and second highpass or bandpass filters 357, 358 can be implemented using simple algorithms that need not match any subsequently utilized image filtering algorithm.

First absolute value extractor 359 receives a first filtered digitized signal from first highpass or bandpass filter 357. Second absolute value extractor 360 receives a second filtered digitized signal from second highpass or bandpass filter 358. The first absolute value extractor 359 extracts absolute values from data contained within the first filtered digitized signal, and the second absolute value extractor 360 extracts absolute values from data contained within the second filtered digitized signal.

A first smoothing and summing mechanism 361 receives absolute values from first absolute value extractor 359. A second smoothing and summing mechanism 362 receives absolute values from second absolute value extractor 360. First and second smoothing and summing mechanisms 361, 362 may each be implemented using an infinite impulse response (IIR) lowpass filter, which effectively functions as a mechanism for summing accumulated absolute values in a manner such that older accumulated values are eventually discarded.

A system control mechanism 363 enables the accumulation of absolute values in the first and second smoothing and summing mechanisms 361, 362 only after a predetermined amount of time has elapsed subsequent to first and second transducer elements 351, 352 transmitting an ultrasound signal. The system control mechanism 363 then disables first and second smoothing and summing mechanisms 361, 362 prior to an immediately successive transmission of an ultrasound signal by first and second transducer elements 351, 352. Illustratively, system control mechanism 363 may, but need not, be combined with a receive beamformer 365, such that the system control mechanism and the beamformer are implemented by the same application-specific integrated circuit (ASIC). While first and second transducer elements 351, 352 are disabled, any absolute values received by these transducer elements will not be accumulated. However, first and second smoothing and summing mechanisms 361, 362 will hold their present accumulated values while disabled.

The overall magnitudes of the accumulated absolute values in first and second smoothing and summing mechanisms 361, 362 is dependent upon the strength of ultrasound echoes received by first and second transducer elements 351, 352. The accumulated absolute values will exhibit some variance due to the statistical nature of the data. A cross-channel smoothing and normalization mechanism 364 receives accumulated absolute values from the first and second smoothing and summing mechanisms 361, 362. For each of the first and second channels 341, 342, cross-channel smoothing and normalization mechanism 364 converts the accumulated absolute values into a set of per-channel apodization values. The per-channel apodization values are determined in a manner so as to have a maximum value of approximately 1.0. In practice, there are relatively small differences between apodization values determined using adjacent channels on the transducer array. The per-channel apodization values constitute an estimate of aperture shading.

The per-channel apodization values are fed to a multiplier 367 which multiplies the apodization values by receive data contained within digitized data received from first and second analog to digital converters 355, 356. This multiplication step is performed prior to beamforming, so as to improve the signal-to-noise ratio of the digitized data from the first and second analog to digital converters 355, 356. The cross-channel processing performed by cross-channel smoothing and normalization mechanism 364 may be implemented periodically, under the control of an update signal received from system control mechanism 363.

Cross-channel smoothing and normalization mechanism 364 calculates the centroid of the aperture shading estimate. The centroid is forwarded to receive beamformer 365, whereupon the beamformer uses the centroid to shift a receive focal point of the transducer array from a first location to a second location. In this manner, a plurality of continually-focused receive beams will originate and be steered in substantially the same way as the shaded transmit beam, to greatly reduce the sensitivity imbalance between multiple receive beams in situations where an occlusion shades the aperture of the transducer array. The foregoing method of estimating the centroid is more direct and accurate than the prior art technique of using data processed by a beamformer.

The methods and systems previously described in conjunction with FIGS. 12 and 13 may be further understood with reference to FIGS. 3-7. FIGS. 3-7 set forth five illustrative cases of aperture shading and the resulting roundtrip beam profiles in an operational environment of four-way parallel beam forming. In each case, the beam plots were taken at a depth of 8 cm with a transmit focus of 4 cm, and a heavily apodized aperture was employed to broaden the transmit beam so as to allow four-way parallel beam forming. TABLE 1 (below) illustrates the effect on roundtrip beam intensity for each of 4 angles of interrogation, and for five different aperture shading (or blockage) situations. Case 3, depicted in FIG. 5, represents an expected 37 symmetric about center” pattern associated with no aperture blockage. The effects summarized in TABLE 1 and illustrated in FIGS. 3-7 would be more pronounced with an integrated measurement over multiple depths beyond the transmit focus.

TABLE 1
Aperture Blocking/Shading Effects

| | | Portion of Physical | Portion of Active | Rec. 1 Peak | Rec. 2 Peak | Rec. 3 Peak | Rec. 4 Peak |
|---|---|---|---|---|---|---|---|
| Case | FIG. | Aperture Blocked | Aperture Blocked | Amp. (Volt) | Amp. (Volt) | Amp. (Volt) | Amp. (Volt) |
| 1 | 3 | Right ⅓ | ⅙ | 18 | 24 | 28 | 29* |
| 2 | 4 | Right ¼ | ⅛ | 24 | 29 | 31* | 30 |
| 3 | 5 | None | None | 28 | 31* | 31* | 28 |
| 4 | 6 | Left ¼ | ⅛ | 30 | 32* | 29 | 24 |
| 5 | 7 | Left ⅓ | ⅙ | 29* | 28 | 24 | 19 |

*represents location of transmit beam maximum

As is apparent from the results set forth in TABLE 1 considered in conjunction with FIGS. 3-7, the occlusion of the active aperture by the ribs results in amplitude modulation of the receive beams. Once the nature of the occlusion is determined, various correction schemes, comprising both static and dynamic correction schemes, can be employed to compensate for the presence of the occlusion.

1. Static Correction Schemes

Pursuant to one static correction scheme that may be employed in accordance with the teachings herein, after the extent of occlusion has been determined, the system turns off both the receive and transmit channels determined to be occluded. The receive focusing parameters are then adjusted so that the center channel focusing coefficients are aligned with the new center of the aperture. Hence, if it is determined that a portion of the active aperture is occluded, the active aperture can be translated over so that it is re-centered about the non-occluded portion of the original active aperture, after which scanning can resume. This process effectively redefines the active aperture by making appropriate steering angle adjustments, and the transmit beam is focused from the new active aperture. TABLE 2 illustrates how such a process might be implemented for the five detection cases mentioned above and illustrated in FIGS. 3-7.

TABLE 2
Static Correction Scheme

| | | Channels | | Extent of |
|---|---|---|---|---|
| | | Turned | Direction of | Realignment (# |
| Case | FIG. | Off | Realignment | of channel positions) |
| 1 | 3 | 85-127 | Left | 10 |
| 2 | 4 | 96-127 | Left | 5 |
| 3 | 5 | None | None | None |
| 4 | 6 | 0-31 | Right | 5 |
| 5 | 7 | 0-41 | Right | 10 |

By realigning the active aperture, “line-to-line” amplitude modulation artifacts are eliminated, because the modified receive beams now track the transmit beam correctly. Consequently, image brightness is much more uniform. Moreover, any drop in resolution comes from the aperture occlusion itself, not from the realignment of the focus. Hence, this correction scheme does not itself result in any further loss of image resolution. By contrast, the conventional approach of using lateral blending filters to reduce amplitude differences between lines (thereby producing an image of more uniform brightness and eliminating image striations) results in loss of image resolution in addition to the loss caused by the occlusion itself.

Several variations on this approach are possible in accordance with the teachings herein. For example, re-alignment of the receive focusing could be done by re-mapping the receive focusing coefficients to receiver channel assignments. Alternatively, the receive focusing coefficients could be pre-calculated for various states of occlusion, stored off-line, and then accessed as needed.

2. Dynamic Correction Scheme

In the methods described above, the correction scheme is static. That is, the probe is placed over the area to be imaged, and adjustments are made if an occlusion is present. These adjustments may occur automatically, or through a suitable prompt (e.g., by pressing a button on the probe). In many instances, however, a dynamic scheme is required. For example, since the sonographer typically moves the transducer array somewhat continuously during an exam, it is desirable to be able to handle movement from a fully open acoustic window (i.e., no occlusion) to a partially blocked acoustic window, and then either back to a fully open acoustic window or to a more severely blocked acoustic window.

Various dynamic adaptive algorithms can be employed in accordance with the teachings herein to account for the presence of occlusions in such situations. Some of these dynamic algorithms are adaptations of the static schemes described herein.

For example, in the 4-way case illustrated in FIGS. 3-7, the correction algorithm can simply constrain itself to moving from one case (of a static correction scheme) to the next in a continuous, closed loop format. Some hysteresis may be employed to ensure smooth imaging. Consequently, the steady state in such a scheme would be the case illustrated in FIG. 3, in which the amplitudes are symmetric about the optical axis.

In both static and dynamic correction schemes, if receive channels are turned off corresponding to the estimated aperture shading, the channels are turned off at a point in the signal path after the aperture shading estimation. Thus all receive channels are used for aperture shading estimation, even if only a subset of receive channels are used for beamforming and image construction.

3. Extension for Non-Parallel Systems

The same misalignment of the transmit and receive beams also occurs in the non-parallel case (that is, in cases where multiple receive beams are not arranged in parallel), although it does not typically manifest itself with the “line-to-line” artifact that occurs in the image when a parallel beamformer is used. Instead, the misalignment is manifested as a drop in amplitude of the roundtrip beam away from the transmit focus and by the creation of an asymmetric side lobe pattern. This effect is illustrated in FIG. 8.

Since the aperture shading estimation and compensation operates on the transmit and receive channels rather than on beamformed data, its operation is not affected by whether there is parallel receive beamforming or not.

4. Extension to 3-D Imaging with a Matrix Transducer

The methods disclosed herein have principally been described with reference to 2-D imaging. However, these methods may be readily adapted to 3-D imaging. To do so, the detection scheme would need to track the rib placement in 2 dimensions, and the correction scheme would need to shift the center of the receiver focus in 2 dimensions as well.

FIG. 9 illustrates a 4-way parallel beam pattern 301 that can be employed in 3-D imaging. The beam pattern includes a transmit beam 303 and receive beams 305, 307, 309 and 311. Since the aperture shading estimation and compensation operates on the transmit and receive channels rather than on beamformed data, its operation is not affected by whether the beams are steered in two dimensions or three dimensions. However, the cross-channel smoothing of the aperture shading estimation needs to take into account whether the channels are arranged in a one dimensional or two dimensional array. In most other respects, a static or dynamic correction scheme employed in a 3-D setting would be conceptually similar to a scheme employed in a 2-D setting.

FIG. 10 shows a simplified block diagram of one possible ultrasound imaging system 10 that may be used in the implementation of the methodologies disclosed herein. It will be appreciated by those of ordinary skill in the relevant arts that the ultrasound imaging system 10, as illustrated in FIG. 10, and the operation thereof as described hereinafter, is intended to be generally representative of such systems and that any particular system may differ significantly from that shown in FIG. 10, particularly in the details of construction and in the operation of such system. As such, the ultrasound imaging system 10 is to be regarded as illustrative and exemplary, and not limiting, as regards the methodologies and devices described herein or the claims attached hereto.

The ultrasound imaging system 10 generally includes an ultrasound unit 12 and a connected transducer 14. The transducer 14 includes a spatial locator receiver 16. The ultrasound unit 12 has integrated therein a spatial locator transmitter 18 and an associated controller 20. The controller 20 provides overall control of the system by providing timing and control functions. The control routines include a variety of routines that modify the operation of the receiver 16 so as to produce a volumetric ultrasound image as a live real-time image, a previously recorded image, or a paused or frozen image for viewing and analysis.

The ultrasound unit 12 is also provided with an imaging unit 22 for controlling the transmission and receipt of ultrasound, and an image processing unit 24 for producing a display on a monitor (See FIG. 11). The image processing unit 24 contains routines for rendering a three-dimensional image. The transmitter 18 is preferably located in an upper portion of ultrasound unit 12 so as to obtain a clear transmission to the receiver 16. Although not specifically illustrated, the ultrasound unit described herein may be configured in a cart format.

During freehand imaging, a technician moves the transducer 14 over the subject 25 in a controlled motion. The ultrasound unit 12 combines image data produced by the imaging unit 22 with location data produced by the controller 20 to produce a matrix of data suitable for rendering onto a monitor (see FIG. 11). The ultrasound imaging system 10 integrates image rendering processes with image processing functions using general purpose processors and PC-like architectures. On the other hand, use of ASICs to perform the stitching and rendering is possible.

FIG. 11 is a block diagram 30 of an ultrasound system that may be used in the practice of the methodologies disclosed herein. The ultrasound imaging system shown in FIG. 11 is configured for the use of pulse generator circuits, but could be equally configured for arbitrary waveform operation. The ultrasound imaging system 10 uses a centralized architecture suitable for the incorporation of standard personal computer (“PC”) type components and includes a transducer 14 which, in a known manner, scans an ultrasound beam, based on a signal from a transmitter 28, through an angle. Backscattered signals or echoes are sensed by the transducer 14 and fed, through a receive/transmit switch 32, to a signal conditioner 34 and, in turn, to a beam former 36. The transducer 14 includes elements which are preferably configured as a steerable, two-dimensional array. The signal conditioner 34 receives backscattered ultrasound signals and conditions those signals by amplification and forming circuitry prior to their being fed to the beam former 36. Within the beam former 36, ultrasound signals are converted to digital values and are configured into “lines” of digital data values in accordance with amplitudes of the backscattered signals from points along an azimuth of the ultrasound beam.

The beam former 36 feeds digital values to an application specific integrated circuit (ASIC) 38 which incorporates the principal processing modules required to convert digital values into a form more conducive to video display that feeds to a monitor 40. A front end data controller 42 receives lines of digital data values from the beam former 36 and buffers each line, as received, in an area of the buffer 44. After accumulating a line of digital data values, the front end data controller 42 dispatches an interrupt signal, via a bus 46, to a shared central processing unit (CPU) 48. The CPU 48 executes control procedures 50 including procedures that are operative to enable individual, asynchronous operation of each of the processing modules within the ASIC 38. More particularly, upon receiving an interrupt signal, the CPU 48 feeds a line of digital data values residing in a buffer 42 to a random access memory (RAM) controller 52 for storage in random access memory (RAM) 54 which constitutes a unified, shared memory. RAM 54 also stores instructions and data for the CPU 48 including lines of digital data values and data being transferred between individual modules in the ASIC 38, all under control of the RAM controller 52.

The transducer 14, as mentioned above, incorporates a receiver 16 that operates in connection with a transmitter 28 to generate location information. The location information is supplied to (or created by) the controller 20 which outputs location data in a known manner. Location data is stored (under the control of the CPU 48) in RAM 54 in conjunction with the storage of the digital data value.

Control procedures 50 control a front end timing controller 45 to output timing signals to the transmitter 28, the signal conditioner 34, the beam former 36, and the controller 20 so as to synchronize their operations with the operations of modules within the ASIC 38. The front end timing controller 45 further issues timing signals which control the operation of the bus 46 and various other functions within the ASIC 38.

As previously noted, control procedures 50 configure the CPU 48 to enable the front end data controller 44 to move the lines of digital data values and location information into the RAM controller 52, where they are then stored in RAM 54. Since the CPU 48 controls the transfer of lines of digital data values, it senses when an entire image frame has been stored in RAM 54. At this point, the CPU 48 is configured by control procedures 50 and recognizes that data is available for operation by a scan converter 58. At this point, therefore, the CPU 48 notifies the scan converter 58 that it can access the frame of data from RAM 54 for processing.

To access the data in RAM 54 (via the RAM controller 52), the scan converter 58 interrupts the CPU 48 to request a line of the data frame from RAM 54. Such data is then transferred to a buffer 60 associated with the scan converter 58 and is transformed into data that is based on an X-Y coordinate system. When this data is coupled with the location data from the controller 20, a matrix of data in an X-Y-Z coordinate system results. A four-dimensional matrix may be used for 4-D (X-Y-Z-time) data. This process is repeated for subsequent digital data values of the image frame from RAM 54. The resulting processed data is returned, via the RAM controller 52, into RAM 54 as display data. The display data is typically stored separately from the data produced by the beam former 36. The CPU 48 and control procedures 50, via the interrupt procedure described above, sense the completion of the operation of the scan converter 58. The video processor 62 interrupts the CPU 48 which responds by feeding lines of video data from RAM 54 into the buffer 62, which is associated with the video processor 64. The video processor 64 uses video data to render a three-dimensional volumetric ultrasound image as a two-dimensional image on the monitor 40.

The above description of the invention is illustrative, and is not intended to be limiting. It will thus be appreciated that various additions, substitutions and modifications may be made to the above described embodiments without departing from the scope of the present invention. Accordingly, the scope of the present invention should be construed solely in reference to the appended claims.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for reducing line-to-line image distortion in an ultrasound imaging system having an aperture by estimating aperture shading caused by the presence of an occlusion in a test subject, the method comprising the steps of:
transmitting a plurality of transmit beams towards a subject using the ultrasound imaging system; each transmit beam being associated with a plurality of receive beams that are reflected by the subject;
collecting the plurality of receive beams using a plurality of receive channels;
deriving receive data from one or more collected receive beams for each of the plurality of receive channels;
generating a sum of the absolute values of the receive data for each of the plurality of receive channels;
smoothing and normalizing the generated sums of the absolute values for each of the plurality of receive channels, so as to generate an estimate for a shaded aperture function that characterizes aperture shading caused by the occlusion; and
using the shaded aperture function as a receive apodization function to improve the signal-to-noise ratio of the receive data, wherein apodization refers to a process of tapering the plurality of receive channel amplitudes using a weighting function.

2. The method of claim 1, further comprising the step of compensating for the occlusion by:
determining a first centroid for the aperture using the shaded aperture function;
deactivating one or more receive channels and one or more transmit channels associated with an occluded portion of the aperture using the shaded aperture function;
determining a second centroid for the aperture; and
using the second centroid to adjust the paths of any receive channels that have not been deactivated.

3. The method of claim 2, wherein the step of using the second centroid to adjust the paths of one or more of the receive beams is performed by determining each of a plurality of respective delays and magnitude scaling factors to be applied to each of the plurality of corresponding receive channels that have not been deactivated; wherein the respective delays and magnitude scaling factors are determined such that a center channel focusing coefficient specifying a center focus of the plurality of receive channels that have not been deactivated is aligned with the second centroid.

4. The method of claim 3, wherein the one or more receive channels to be deactivated are determined by measuring the magnitudes of amplitude modulation components on each of the plurality of receive channels as a function of relative placement of each of a plurality of receive beams.

5. The method of claim 3, wherein the one or more receive channels to be deactivated are determined by measuring the magnitudes and locations of amplitude modulation components on each of the plurality of receive channels.

6. The method of claim 3, wherein the one or more receive channels to be deactivated are determined by measuring the magnitudes of amplitude modulation components on each of the plurality of receive channels.

7. The method of claim 2, wherein the step of compensating for the occlusion includes the step of refocusing the transmit beams.

8. The method of claim 2, wherein the step of compensating for the occlusion is static.

9. The method of claim 2, wherein the step of compensating for the occlusion is dynamic.

10. The method of claim 2, wherein the step of compensating for the occlusion is performed by moving the centroid of the aperture from a first location to a second location.

11. The method of claim 10, further comprising the step of monitoring each of the receive beams to identify any receive beam that is shaded by an occlusion.

12. The method of claim 11 wherein the ultrasound imaging system includes one or more aperture elements, each aperture element being associated with a receive channel, the method further comprising the step of deactivating one or more receive channels associated with any aperture element shaded by an occlusion.

13. The method of claim 12, further comprising the step of realigning a focal point of the receive beams such that at least one receive channel is aligned with the new center of the aperture.

14. The method of claim 12, wherein the receive channels associated with any aperture elements shaded by an occlusion are identified by determining an amount and a location of an amplitude modulation component relative to placement of the receive beams.

15. An ultrasound imaging device for estimating the extent of any occlusion of the aperture and compensating for the estimated occlusion, the imaging device comprising:
a transducer array, having an aperture associated therewith, and including a plurality of aperture elements for emitting acoustic pulses over a plurality of transmit channels, and for receiving analog echoes of these pulses over a plurality of receive channels;
an analog to digital converter, being coupled to the transducer array, for converting the analog echoes into digital receive data;
a bandpass or highpass filter for filtering the digital receive data to be utilized for aperture shading estimation by deemphasizing lower frequencies;
an absolute value extractor for extracting absolute values from the filtered digital receive data;
a smoothing mechanism for smoothing the extracted absolute values; and
a normalization mechanism for normalizing the smoothed extracted absolute values from a plurality of receive channels to generate an estimate of the extent of any occlusion of the aperture in the form of an apodization function used by the transducer array to improve the signal-to-noise ratio of the receive data.

16. The ultrasound imaging device of claim 15, wherein the normalization mechanism is equipped to determine the centroid of the apodization function.

17. The ultrasound imaging device of claim 16, further comprising a beamformer, wherein the determined centroid is utilized in conjunction with the beamformer to adjust the paths of one or more of the receive beams, so as to compensate for the occlusion.

18. The ultrasound imaging device of claim 17, wherein the beamformer is equipped to:
(a) monitor each of the receive channels so as to determine the extent of an occlusion that moves the original center of the aperture to a new center;
(b) deactivate the receive channels associated with any aperture elements blocked or shaded by an occlusion; and
(c) re-align the receive focusing such that the center receive channels are aligned with the new center of the aperture.
